(19) United States
(12) Patent Application Publication
(10) Pub. No.: US 2010/0173271 A1
(43) Pub. Date: Jul. 8, 2010

(54) Computer operated by thought and emotions

(76) Inventor: Ruchit Kumar Regmi, Kathmandu (NP)

Correspondence Address: Ruchit Kumar Regmi, GPO Box 23890, Kathmandu, NP

(21) Appl. No.: 12/378,026

(22) Filed: Jan. 6, 2009

(51) Int. Cl. G09B 19/00; A61B 5/04

(52) U.S. Cl. 434/236; 600/544

(57) ABSTRACT

My innovated computer doesn't need input devices like keyboards, mouse, light pen, etc; rather, it needs head cap in the form of comfortable headset to sense and to detect the instantaneous electrical signals generated in the brain according to the thought and emotions of the computer user as an input. The usage field of computer is as PC, Computer uses in: Investigation Purposes, Psychology, Neuro-psychiatry, Neurology. After the production of the computer Computing, information, communication and investigation fields will be revolutionized.

This computer system uses thought and emotion of the brain for the input. By using human anatomy of brains and its relation with electrical signals and its use in computer engineering such a type of computer can be developed by applying this new innovative idea that can be used in neurology, neuro-psychiatry, psychology, investigation purposes, and in general computer application like personal computer (PC).That means area and claims of this invention will be in the fields of:

(1) Personal computer (PC)

(2) Investigation Purposes

(3) Psychology

(4) Neuro-psychiatry

(5) Neurology

In this new system, input to the Central Processing Unit (CPU) is provided by the human brain (and can also from any part of the body if modified). In human brain, very small electrical signal (pulses) produces according with our thought and emotions. According to our thoughts' and emotions' intensity and type, the instantaneous electrical signal (within brain) with different amplitude (intensity) and in different frequencies induces. By detecting those electrical signals and amplifying (if necessary) and transferring, and then giving input to the CPU of the computer; we can achieve output after coding/decoding (transformation/transmission) and other processing and procedures within the computer hardware (just like in present computer). According with the instantaneous electrical signals (pulses) detected (sensed) from the brain according with our thoughts and emotions. For example, if in the present PC from typing the character “A” let us assume the pulse of high high low comes after conversion from the instantaneous signal and this high high low electrical pulses will be feed upto the CPU of the computer and after processing, coding/decoding output can be retrieved; at the same way in my innovated computer while operator (computer user) thinks “A” at his certain emotional state, the instantaneous electrical signals will be generated in the brain according with the thinking at the certain emotional state and those signal will be converted into binary pulse code. Let us assume at highly emotional state computer operator thought “A” then high low low binary coded pulse signal generated then the signal has to feed up to the CPU of the computer and those high low low binary coded pulse signal has to be interpreted, has to be transformed and coding/decoding should be done to get the same output as the output retrieved after pressing the character “A” on the keyboard of the present PC. That means, conversion and interpretation are needed to do the same job of present PC from my invented computer.

However, present PC needs keyboard, mouse, light pen etc as the input devices but my computer doesn't need such type of input devices rather it needs head cap in the form of adjustable headset to sense and to detect the electrical signals generated in the brain according with our thought and emotions.

The signal detecting system from our brain will be just like (but not exactly) by EEG (Electroencephalogram). In EEG, by connecting electrodes (sensors) in the different points we can detect different brain waves and signals according with our thought and emotions and can be viewed graphically. But in my detecting system, a little difference occurs. Electrical signals sensing and detecting elements can be in the form of cap (head cap) like that of adjustable head set for the detection of electrical signals through the brains. To transfer the instantaneous electrical signal (pulses) detected from the brain according with the thought and emotions will have to be in standard and uniform form to feed up to the CPU of the computer. Position of the detecting sensor will vary according to the products' usage fields; and coding/decoding within the CPU should be done according to the purposes and the fields of application of computer.

Coding means converting input signals into machine/assembly code (language) and decoding means converting machine/assembly code (language) into output. After detecting electrical signals (pulses) from the head cap, those electrical signals can be transformed in any relevant type of energy as required. In my computer, those electrical signals is are transferred either by wire and or by wireless (radio) to the CPU.

This innovation will revolutionize the world's information, communication, computing fields radically and also will help in medical and investigation field a lot.

Supplementary Details:

The computer used in the claim field of personal computer (PC), for PC manufacturing purposes guided/directed conscious thought and emotion will be taken as the reference signals/pulses for the input of the devices. Those references will be taken for prototype development, product development and product manufacturing.

According to the thought and emotions, the instantaneous electrical signals/pulses can be detected and those signal/pulses will be used for the input of the devices (my innovated computer) for manufacturing purposes by taking the ideas as references.

In the brains, electrolyte levels will be changed continuously according to the thought and emotions. Due to the changes in the flow, level, and type of charged particles changes within the brain including in the neurons, veins etc of the nervous systems those instantaneous changes in the electrolytes/charged particles or the changes in the deposition of ions will become varied instantaneously according to the thought and emotions. Those instantaneous changes in the type and level of the charged particles will create potential differences within the brains including in the neurons, veins etc of the nervous system. Those rate, level and type of changes in the deposition of ions/charged particles and the potential differences created by the changes will be analyzed, processed and detected in the tiny electrical pulses/signal form for the input of the devices at the reference of the ideas for manufacturing purposes. Those detected instantaneous electrical signal/pulses will be analyzed, processed according to the claim usage fields of the computer in the different field of claims: personal computer (PC), computer uses in investigation purposes/psychology/neuro-psychiatry/neurology. After the detection, analysis and its processing, the detected signals will be amplified according to its necessity and will be processed for the input of the related claim fields of the devices. During signal processing, the usage claim fields will be taken into notices and consideration then following the process, processed signals will be made in standard digital forms (in digital form because in analog form it may be more complicated to bypass unessential signals/pulses i.e. noises). i.e. in the uniform digitally coded standard form.

All other unrelated (not essential) signals/pulses that is electronic noises of the related devices will be made bypassed, and only the related (essential) signals/pulses after and during the processing will be used in the related claim fields of the devices (computer) as the reference ideas for the manufacturing purposes.

The head cap in the form of adjustable head set will be at least comfortable size and shape. During signal detection and head set manufacturing the permitted range for good health will be taken into consideration even if the head set is used for the long time in the long run by the computer user.

The usage type of thought and emotions for the input of the computer (devices) will be as guided/directed conscious thought and emotions and or unguided/undirected or random conscious and or unconscious (sub conscious) thought and emotions. Those type of thought and emotions which will be used for the input of the devices will be determined according to the purposes and application field of the computer, usage purposes/application fields and fields claimed in the invention. The type of usage of the thought and emotions and the detecting signals/pulses will be selected and determined for the input of the devices during and after the detection of the signals/pulses from the brain, and other unessential (not necessary) signals/pulses that is noise will be bypassed.

For example, devices (computer) used in the investigation purposes: the guided/directed and random (undirected/unguided) conscious thought and emotions will be used at the reference ideas during manufacturing the devices. For the computer used in psychology, neuro-psychiatry claimed fields: directed/guided and random (unguided/undirected) unconscious (subconscious) thought and emotion will be taken as the references for the input in the device manufacturing; however, claim fields in the investigation field, psychology, and neuro-psychiatry field, one another type of the devices can be used for further diagnosis and investigation. For the computer claimed in neurology fields: all type of signals i.e. random (unguided/undirected) and directed (guided) conscious and unconscious (subconscious) signals/pulses will be processed; and according to the type, strength, level of the process signal/pulses and output of the devices the flow type, levels, other features and parameters of the fluids in the neurons, veins, brains and in the nervous systems will be determined after the processing of the signals and from the output of the computer. Output of the computer can be made easily understood for the related users. For the manufacturing process, all other related and essential processing shall be taken as referenced ideas from the idea mentioned in the specification (theory of operation) of the title of this invention “Computer operated by thought and emotions”, application Ser. No. 60/625969 of my provisional applications and application Ser. No. (ASN): 12/378026 of my non provisional applications. Therefore, after the production of the devices not only the radical changes will take place in the computing, communication, information technology fields but by using this type of computer as mentioned in the claims of investigation and psychology fields investigation can be made thoroughly to the suspected terrorist and criminals; as a result, terrorism can be controlled and numbers of crimes also can be controlled.

1. Computer uses as :
(1) Personal Computer (PC)
(2) In Investigation Purposes
(3) In Psychology
(4) In Neuro-psychiatry
(5) In Neurology
